(19) United States
(12) Patent Application Publication
(10) Pub. No.: US 2020/0405999 A1
(43) Pub. Date: Dec. 31, 2020

(54) HOLDER AND AROMATHERAPY COMPONENT

(71) Applicant: Sherie Goodstadt, Brookville, NY (US)

(72) Inventor: Sherie Goodstadt, Brookville, NY (US)

(21) Appl. No.: 16/946,563

(22) Filed: Jun. 26, 2020

Related U.S. Application Data
(60) Provisional application No. 62/866,999, filed on Jun. 26, 2019.

(51) Int. Cl. A61M 21/02; B01D 53/04; A47G 9/00; A47G 9/02; A47G 9/10

(52) U.S. Cl. CPC A61M 21/02; B01D 53/04; A47G 9/007; A61M 2021/0016; A47G 9/1045; B01D 2253/102; A47G 9/0253

(57) ABSTRACT

The present technology relates to an item cover for containing an aromatherapy component for providing an aromatherapy. The item cover includes an exterior and an interior including an inner lining. The interior receives a stuffed item. The item cover further includes at least one pocket for receiving at least one removeable insert comprising an aromatherapy component.

### CROSS-REFERENCE TO RELATED APPLICATIONS

This application claims the benefit of the filing date of U.S. Provisional Patent Application No. 62/866,999 filed Jun. 26, 2019, the disclosure of which is hereby incorporated herein by reference.

### TECHNICAL FIELD

The present technology concerns items for aromatherapy such as an item cover with one or more insertable and/or removable scented components. In some versions, a pillow case may include a scented component, such as with essential oil remedy blends. The scented component may be a material configured for receiving one or more scents, such as by infusion of one or more a scented blend of oil(s). The scented component may be for example, a rubber material, and may configured as an insertable component that may be received in a component housing of the item cover. Thus, the insertable component may be replaced with other scented inserts for changing scents and/or renewing a scented component when depleted.

### BACKGROUND OF THE INVENTION

Millions of people are exploring natural solutions to their health problems and/or ways to just feel good, sleep better, reduce stress and gain overall wellness. One in three adults do not get enough sleep and this is when our immune system works the hardest. Thus, some examples of the present technology may employ aromatherapy components for therapeutic release of oils such as to improve health and may in some examples may be configured for such a release, such as during sleep or rest.

### BRIEF SUMMARY OF THE INVENTION

The present technology concerns items for aromatherapy such as an item cover with one or more insertable and/or removable scented components.

One aspect of the present technology relates to an item cover for containing an aromatherapy component for providing an aromatherapy comprising: an exterior; an interior including an inner lining, the interior configured to receive a stuffed item; and at least one pocket for receiving at least one removeable insert comprising an aromatherapy component.

According to another aspect of the present technology, the aromatherapy component is infused with one or more oils.

According to another aspect of the present technology, the aromatherapy component is infused with sufficient oils such that the insert releases scents for a predetermined period of time.

According to another aspect of the present technology, the infusion is one of cold infusion or heat infusion.

According to another aspect of the present technology, the infusion is performed at room temperature.

According to another aspect of the present technology, the infusion time is in the range of 6 and 24 hours.

According to another aspect of the present technology, the aromatherapy component is made of rubber.

According to another aspect of the present technology, the rubber is gum rubber.

According to another aspect of the present technology, the aromatherapy component is made of a flexible material.

According to another aspect of the present technology, the aromatherapy component is pre-packaged.

According to another aspect of the present technology, the packaging includes a barrier to contain aroma releasable by the aromatherapy component and is configured to be opened on a single side, such that when the removeable insert is disposed in the at least one pocket, the aromatherapy component releases aroma only from the opened side of the packaging.

According to another aspect of the present technology, the opened side of the packaging is oriented toward the exterior of the item cover.

According to another aspect of the present technology, the packaging is a foil pouch that is discardable before the removeable insert is disposed in the at least one pocket.

According to another aspect of the present technology, the at least one pocket is sewn into the item cover adjacent to the inner lining such that the at least one pocket is hidden when the item cover is in use.

According to another aspect of the present technology, the at least one pocket includes an opening disposed along an edge or seam of the item cover.

According to another aspect of the present technology, the at least one pocket is aroma-permeable.

According to another aspect of the present technology, the at least one pocket includes an aroma permeable side and an aroma-impermeable side.

According to another aspect of the present technology, the aroma-permeable side is oriented toward the exterior of the item cover and the aroma-impermeable side is oriented toward the stuffed item in the interior of the item case.

According to another aspect of the present technology, the item cover includes at least one layer configured to filter undesirable gases released by the stuffed item.

According to another aspect of the present technology, the item is a pillow and the item cover is a pillow case.

### BRIEF DESCRIPTION OF THE DRAWINGS

FIG. 1 is a side view of the exterior of an item cover in accordance with an embodiment of the present disclosure;

FIG. 2 is a side view of the interior of the item cover of FIG. 1 in accordance with an embodiment of the present disclosure;

FIG. 3 is an opposite side view of the interior of the item cover of FIG. 1 in accordance with an embodiment of the present disclosure;

FIG. 4 illustrates a removeable insert inserted into a pocket of the item cover of FIG. 1 in accordance with an embodiment of the present disclosure; and

FIG. 5 is a perspective view of a removeable insert for use with the item cover of FIG. 1 in accordance with an embodiment of the present disclosure.

### DETAILED DESCRIPTION

Embodiments of the present disclosure may be considered in relation to the detail with of the drawing figures. It is to be understood that the disclosed embodiments are merely examples of the disclosure, which may be embodied in various forms, materials, and sizes. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

The drawings illustrate an example aromatherapy item cover which may serve as a pillow cover or pillow case. The example pillow case may include a pocket to house an insertable scent-containing component or insert (e.g., with essential oil remedy blends) that may be infused into the insert (e.g., a rubber material such as gum rubber). The case or cover may otherwise be configured to receive stuffing such as a pillow. The insertable component may be configured of a shape and size that corresponds to the housing of the pillow case, such as an internal pocket of the pillow case.

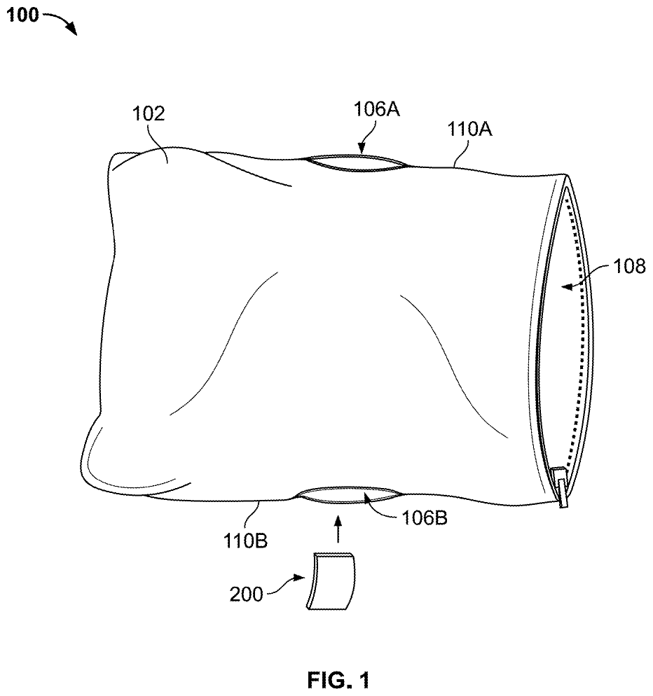

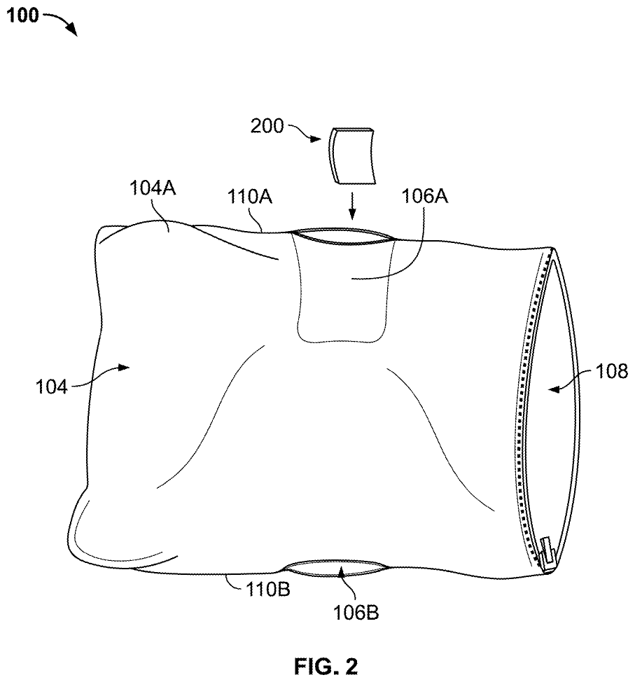

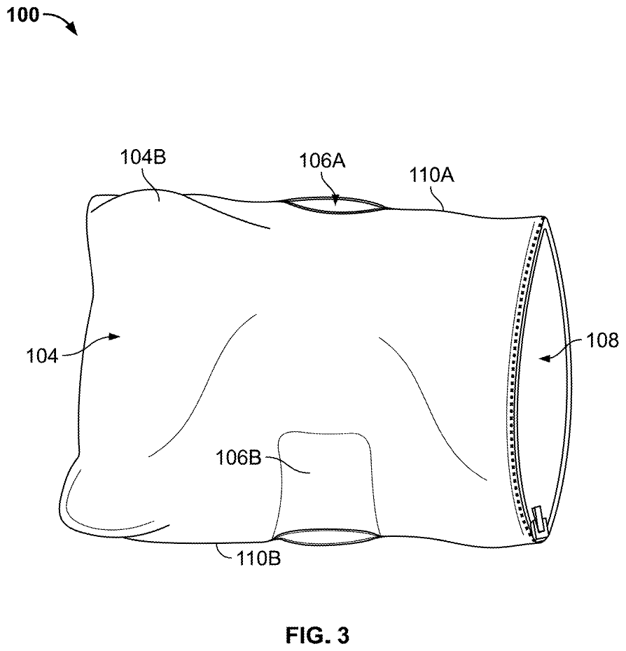

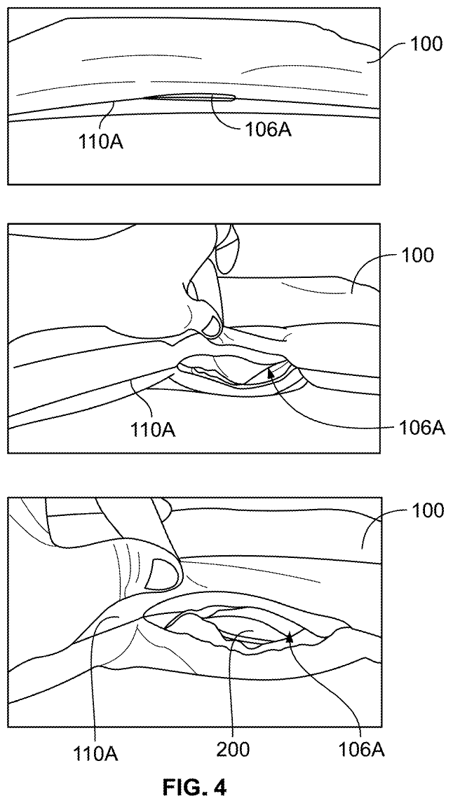

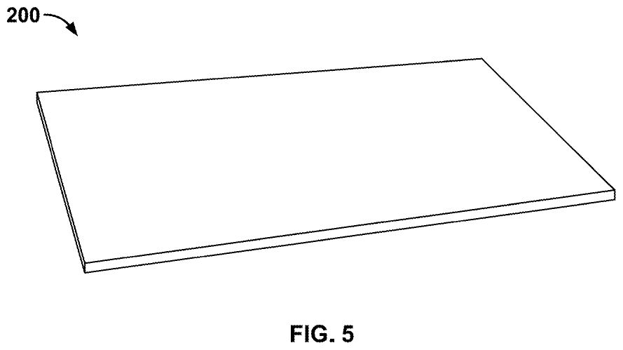

For example, as illustrated in FIGS. 1, 2, 3, 4 and 5, the pillow case or other item cover 100 on which a person may rest, such as during sleep, may have a hidden pocket 106A such as one or more built on or sewn in pocket(s) 106A, 106B to house the rubber infused inserts 200. Thus, the pillow case 100 may house one or more aromatherapy inserts 200. An example insert 200 is shown in FIG. 5. An example case, with pockets 106A, 106B is also shown in FIG. 5 where the hidden pockets 106A, 106B have openings, sized for the insert 200, along an edge or seam 110A, 110B (corresponding to each of pockets 106A, 106B) of the case 100. In some forms, the case or holder 100 may be in the form of or for a neck pillow such that the infused component(s) 200 may be inserted within a pocket(s) 106A, 106B of the neck pillow for use.

A benefit of the present technology, such as with a pillowcase, may be to enhance health and wellness by infusing essential oil blends into interchangeable and removable rubber inserts 200 that can be chosen depending upon each individual's desired remedy. In the example of a pillowcase 100, one or more hidden pockets 106A, 106B can house essential oil remedy blends that may be infused into one or more rubber inserts 200. Thus, different inserts 200 may have different aromatherapy or oil blends.

Each insert 200 may be infused with sufficient oils so that it may release for a desired use period. The infusion may be accomplished in a temperature-controlled environment, such as by cold infusion (e.g., 0-5 degrees Celsius, such as 1 degree or 2 degrees for example, or other range) or heat infusion (e.g., 20-30 degrees Celsius or other range) and may depend on the particular oil or oil blends being infused. Some infusion may be performed at room temperature or without a temperature-controlled environment. For example, infusion times may be approximately 6 to 24 hours and the temperatures may vary. However, other time amount chosen to adjust the release time (useful life) may be provided. The inserts 200 may be suitably packaged for storage such as to contain the aroma from release until the sealed packaging (such as a foil sealed package) is ready for use and opened to be inserted within the hidden pocket 106A, 106B. Thus, the aroma will release over a set period of time (e.g., three to six weeks, one month or other suitable period of time etc.) when desired. The inserts 200 can, for example, be re-ordered on a subscription basis or as needed.

Thus, an infused insert 200 may be pre-packaged, such as in a heat-sealed foil pouch, to preserve the essential oils and prevent evaporation. Such a package serves as a barrier to contain the oil(s). For use, the foil may be opened, such as by pull tab, and may optionally open only on one side (e.g., a top side) of the insert package. Such a package configuration of the insert 200 having a barrier on one side may permit the aroma to release through the pillowcase 100 and not into the pillow. Thus, the package can serve as a seal or barrier to prevent release into the stuffed item of the cover 100 (e.g., a pillow on which the case is covering). Optionally, such a foil pouch can also be opened completely. Thus, in some cases the pocket 106A, 106B may optionally provide a seal/barrier within the pocket 106A, 106B (e.g., foil) so as to provide an aroma-impermeable barrier between the insert 200 and the pillow at the pocket 106A, 106N of the case 100 and thereby direct release of the aroma toward a user. Thus, the pocket 106A, 106B may be formed in part by both an aroma permeable side (an outer or user adjacent side) and an aroma-impermeable side (an inner side further from the user).

Thus, the infused insert 200 may aid in improving health and wellness in a plethora of areas in the body and mind. For example, by infusing the essential oils into the rubber inserts 200, the present technology may permit uses to benefit from many different essential oil blends while sleeping or resting, such as when using the holder of the insert 200 in close proximity to the person (e.g., laying or leaning against the holder).

Accordingly, such an insertable aromatherapy item 200 can provide a sleep support that is all natural, non-toxic and may also aid in sleep and wellness. For example, essential oils are a natural solution to enhance sleep, improve mood, bolster the immune system, reduce anxiety and much more. A user in proximity of the cover 100 with the insert 200 may then breath in these therapeutic essential oil remedies during sleep or rest to aid and bolster overall health and wellness. For example, a pillowcase 100 configured with its built-in hidden pockets 106A, 106B (e.g., two or more, such as four pocket) can each house an insert 200 and the infused essential oil rubber insert(s) 200 can release therapeutic aromas over a set period of time such as a period of sleep or a period of some other type of use. For example, in some versions, the pocket 106A, 106B may be formed in a chair pillow or a case for a chair cushion so that it may receive an insert 200. Similarly, a chair pillow may have a case or otherwise have pockets 106A, 106B to receive inserts 200 for use at work such as where straps of the pillow or case may be applied around a part of the chair (such as the head rest). Thus, the holder 100 and inserts 200 may be used as a remedy for stress and/or to improve focus/concentration etc. while a user is at work and sitting at a desk, for example. Thus, an insert 200 can be infused so as to be configured to release therapeutic aromas for many different uses such as an insomnia blend for a period of sleep, a focus blend for a period of a work day, an anxiety blend for a period of rest etc.

In the example of the figures, a pillowcase 100 may have a fabric forming the case. The case 100 may have an exterior 102 and an opening 108 for receiving the stuffed item (e.g., the pillow) into an interior of the case 100. The interior may include an inner lining 104 and opposite sides 104A, 104B. The case 100, such as with the fabric, may then have one or more built-in pockets 106A, 106B to receive the essential oil infused rubber inserts 200. In some case, an infused rubber insert 200 may range in size from 2-4 inches wide. It may be 3-6 inches long and ⅛- 1/16 thick. Other sizes are suitable when appropriately matched to the similar size of the pocket 106A, 106B of the case. Moreover, although rubber inserts 200 are contemplated, other infusable materials may be employed so as to contain a variety of different essential oil blends/remedies that may aid in overall wellness. Suitable materials may be flexible so as to promote comfort of a user that may contact the insert 200 such as when in a pillow during user contact such as when sleeping, resting, sitting, etc.

In some versions, the holder 100 may have additional layers, such as one or more filter layers. For example, in the version of a pillow case, a layer of activated carbon cloth may be sewn into the pillow case 100. Such a layer may serve as a barrier between the user and a further component introduced into the pillow case 100. For example, with a pillowcase type holder 100, a user may insert a conventional pillow into the pillow case. The filter layer of the pillow case 100 may then serve as a filter to block any off-gassing of undesirable gasses from the inserted pillow 100, such as undesirable gasses from the manufacturing process of the pillow 100. Thus, the filter layer may be positioned to serve as a barrier to the gas of the pillow within the pillow case 100 from passing to the user. However, the filter layer would not prevent the infused insert 200 from releasing the beneficial aromatherapy toward/to the user. In some versions, the filter layer may be an enclosure to contain the pillow so as to prevent or impede it from off-gassing toward the user.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. An item cover for containing an aromatherapy component for providing an aromatherapy comprising:
an exterior;
an interior including an inner lining, the interior configured to receive a stuffed item; and
at least one pocket for receiving at least one removeable insert comprising an aromatherapy component.

2. The item cover of claim 1, wherein the aromatherapy component is infused with one or more oils.

3. The item cover of claim 2, wherein the aromatherapy component is infused with sufficient oils such that the insert releases scents for a predetermined period of time.

4. The item cover of claim 3, wherein the infusion is one of cold infusion or heat infusion.

5. The item cover of claim 3, wherein the infusion is performed at room temperature.

6. The item cover of claim 3, wherein the infusion time is in the range of 6 and 24 hours.

7. The item cover of claim 1, wherein the aromatherapy component is made of rubber.

8. The item cover of claim 7, wherein the rubber is gum rubber.

9. The item cover of claim 1, wherein the aromatherapy component is made of a flexible material.

10. The item cover of claim 1, wherein the aromatherapy component is pre-packaged.

11. The item cover of claim 10, wherein the packaging includes a barrier to contain aroma releasable by the aromatherapy component and is configured to be opened on a single side, such that when the removeable insert is disposed in the at least one pocket, the aromatherapy component releases aroma only from the opened side of the packaging.

12. The item cover of claim 11, wherein the opened side of the packaging is oriented toward the exterior of the item cover.

13. The item cover of claim 10, wherein the packaging is a foil pouch that is discardable before the removeable insert is disposed in the at least one pocket.

14. The item cover of claim 1, wherein the at least one pocket is sewn into the item cover adjacent to the inner lining such that the at least one pocket is hidden when the item cover is in use.

15. The item cover of claim 1, wherein the at least one pocket includes an opening disposed along an edge or seam of the item cover.

16. The item cover of claim 1, wherein the at least one pocket is aroma-permeable.

17. The item cover of claim 1, wherein the at least one pocket includes an aroma permeable side and an aroma-impermeable side.

18. The item cover of claim 17, wherein the aroma-permeable side is oriented toward the exterior of the item cover and the aroma-impermeable side is oriented toward the stuffed item in the interior of the item case.

19. The item cover of claim 1, wherein the item cover includes at least one layer configured to filter undesirable gases released by the stuffed item.

20. The item cover of claim 1, wherein the item is a pillow and the item cover is a pillow case.
